(19) United States
(12) Patent Application Publication
(10) Pub. No.: US 2023/0197191 A1
(43) Pub. Date: Jun. 22, 2023

(54) EARLY DETECTION OF PATHOGENS IN PLANTS

(71) Applicant: BAYER AKTIENGESELLSCHAFT, Leverkusen (DE)

(72) Inventors: Benjamin BUER, Köln (DE); Arwa AL DILAIMI-WENDLER, Leverkusen (DE); Andreas MEHL, Meerbusch (DE); Jochen KLEEMANN, Kerpen (DE)

(21) Appl. No.: 17/924,650

(22) PCT Filed: Apr. 30, 2021
(86) PCT No.: PCT/EP2021/061366

§ 371 Date: Nov. 10, 2022

(30) Foreign Application Priority Data
May 13, 2020 (EP) 20174320.0
Jul. 3, 2020 (EP) 20183910.7

(51) Int. Cl. G16B 20/20; C12Q 1/6869

(52) U.S. Cl. CPC G16B 20/20; C12Q 1/6869; C12Q 2600/156

(57) ABSTRACT

The present invention deals with the early detection of pathogens of plants, and with the identification of signs of resistance of said pathogens to chemical or biological control agents. The subjects of the present invention are a system, a method and a computer-readable medium for early detection of the presence of pathogens near, on or in plants with the aid of sequence analyses and the identification of signs of resistance.

The present invention deals with the early detection of pathogens in, near or on plants or plant parts, and with the identification of signs of resistance of said pathogens to chemical or biological control agents. The subjects of the present invention are a system, a method and a computer-readable medium for early detection of the presence of pathogens in, near or on plants or plant parts with the aid of sequence analyses and the identification of signs of resistance.

The early detection of an attack by a pathogen on a plant is very important. The earlier an attack is detected, the sooner can suitable measures be taken to prevent a disease outbreak and/or a spread and/or to avert damage to the plants.

WO2017/194276 describes a method for detection of plant diseases that is based on the analysis of images of a plant part. However, a plant can have been attacked by a population of pathogens without visible symptoms already being found. Furthermore, the resistance status of the population of pathogens cannot be ascertained by means of such an image analysis.

US20120088696A1 discloses a micro-electrochemical multiplex PCR platform which can be used for amplifying, analysing and quantifying target nucleotides in real-time and can, for example, be used for rapid detection of plant diseases. However, the platform must be specifically adapted on a case-by-case basis for the detection of a specific disease, and use of the platform is comparatively complex.

It would be advantageous if were possible not only for pathogens to be detected early, but also for resistance to a control agent in the population of pathogens to be already ascertained in an early status.

WO2019149626A1 deals with the identification of resistance markers in a field.

The present invention supplements the solution described in WO2019149626A1. The present invention combines the ascertainment of resistance with early detection of pathogens in plants. The solutions presented in this description are efficient and adaptable and can incorporate further information in the early detection of pathogens and resistance.

In a first aspect, the present invention provides a method comprising the steps of:
collecting a sample of a plant or of a medium in which the plant is growing,
identifying a pathogen in the sample,
identifying signs of a resistance of the pathogen to a control agent,
   wherein the identification of the pathogen and the identification of the signs of a resistance is done by means of sequencing of individual nucleic acids or individual peptides in the sample, wherein sequences of nucleotides or sequences of amino acids are ascertained in the sequencing of the individual nucleic acids or individual peptides, wherein the sequences of nucleotides or sequences of amino acids are compared with reference sequences, wherein the sequencing is done in two phases, a first phase and a second phase,
   wherein, in the first phase, during the sequencing of each nucleic acid or each peptide, the respectively ascertained sequence is compared with at least one pathogen sequence, and
      if the ascertained sequence does not match the at least one pathogen sequence, further sequencing of said nucleic acid or said peptide is aborted,
      if the ascertained sequence matches a pathogen sequence, a pathogen having the pathogen sequence is ascertained and the ascertained pathogen is used to ascertain at least one resistance marker, and a switch is made to the second phase,
   wherein, in the second phase, sequences of the nucleic acids or peptides in the sample are compared with the at least one ascertained resistance marker and/or with a resistance-free sequence.

The present invention further provides a system comprising
a sequencing unit,
a control and calculation unit and
at least one data store in which at least one pathogen sequence and at least one resistance marker are stored for at least one pathogen,

wherein the sequencing unit is configured to sequence nucleic acids or peptides in a sample of a plant or of a medium in which the plant is growing and to thereby ascertain sequences of nucleotides or amino acids in the sample,

wherein the sequencing unit is configured to transmit, during the ascertainment of the sequences, the respectively ascertained sequences to the control and calculation unit,

wherein the control and calculation unit is configured to compare, in a first phase, each transmitted sequence of a nucleic acid or a peptide with at least one pathogen sequence, and
if a transmitted sequence does not match the at least one pathogen sequence, to prompt the sequencing unit to abort further sequencing of the respective nucleic acid or the respective peptide,
if a transmitted sequence matches a pathogen sequence, to ascertain a pathogen having the pathogen sequence and to use the ascertained pathogen to ascertain at least one resistance marker, and to make a switch to a second phase,

wherein the control and calculation unit is configured to compare, in the second phase, sequences of the nucleic acids or peptides in the sample with the at least one ascertained resistance marker and/or a resistance-free sequence.

The present invention further provides a computer-readable (storage) medium comprising commands which, upon execution by a computer, prompt said computer to execute the following steps:
for a multiplicity of nucleic acids or peptides: receiving a growing succession of nucleotides or amino acids in the respective nucleic acid or the respective peptide from a sequencing unit,
in a first phase while the growing succession is being received: checking whether the succession of nucleotides or amino acids is singly or multiply present in at least one pathogen sequence,
if the succession is not present in any pathogen sequence: transmitting a signal to the sequencing unit to abort further sequencing of the nucleic acid or the peptide,
if the succession matches a pathogen sequence: ascertaining a pathogen having the pathogen sequence, and ascertaining at least one reference marker, and initiating a second phase,
in the second phase: comparing the successions of nucleotides or amino acids with the at least one reference marker and/or a resistance-free sequence.

The present invention further provides a computer program comprising commands which, upon execution of the computer program by a computer, prompt the computer to execute the following steps:
for a multiplicity of nucleic acids or peptides: receiving a growing succession of nucleotides or amino acids in the respective nucleic acid or the respective peptide from a sequencing unit,
in a first phase while the growing succession is being received: checking whether the succession of nucleotides or amino acids is singly or multiply present in at least one pathogen sequence,
if the succession is not present in any pathogen sequence: transmitting a signal to the sequencing unit to abort further sequencing of the nucleic acid or the peptide,
if the succession matches a pathogen sequence: ascertaining a pathogen having the pathogen sequence, and ascertaining at least one reference marker, and initiating a second phase,
in the second phase: comparing the successions of nucleotides or amino acids with the at least one reference marker and/or a resistance-free sequence.

Preferred embodiments of the present invention are found in the dependent claims, in the present description and in the drawings.

The invention will be more particularly elucidated below without distinguishing between the subjects of the invention (method, system, computer-readable medium, computer program). Instead, the elucidations that follow are intended to apply analogously to all the subjects of the invention, irrespective of the context (method, system, computer-readable medium, computer program) in which they occur.

If steps are stated in an order in the present description or in the claims, this does not necessarily mean that the invention is restricted to the stated order. Instead, it is conceivable that the steps can also be executed in a different order or else in parallel with one another, unless one step builds on another step, which absolutely requires that the step building on the previous step be executed subsequently (which will however become clear in the individual case). The orders stated are thus preferred embodiments of the invention.

The present invention provides means which allow early detection by a user of an attack on a plant by pathogens and of signs of a resistance of the pathogens to a control agent.

The term “pathogen” is understood to mean microorganisms or subcellular agents which cause processes detrimental to health in other organisms. Pathogens can, for example, be algae, bacteria, parasites, fungi, prions, protists, viruses or viroids. In this description, the term “infectious agent” is used as a synonym for the term “pathogen”. The present invention gives preference to dealing with the early detection of plant-damaging fungi.

The term “control agent” is understood to mean an agent by means of which pathogens can be controlled in an effective manner and/or the spread thereof can be prevented. Examples of control agents are fungicides, insecticides and herbicides. A control agent usually comprises one or more active ingredients. “Active ingredients” are chemical or biological substances which have a specific effect or cause a specific reaction in a pathogen.

The term “resistance” is understood to mean a property of individual pathogens of a species that is shown by the fact that these individuals survive a treatment with a control agent which can normally control the species, and can complete their development cycle as normal or spread further.

The term “user” is understood to mean a person who carries out the invention; in particular, the user is a person who carries out the invention by using the system according to the invention and/or using a computer on which the computer program according to the invention has been installed.

A distinction can be made between two types of resistance: target-specific resistance, which is also called target site resistance, and metabolic resistance, which is also called non-target site resistance.

A target-specific resistance is usually caused by a mutation in an individual gene which is associated with the target of a control agent in the pathogen. The mutation can, for example, bring about stronger expression of the target protein or an alteration of the site to which the active ingredient of the control agent binds. There are molecular biology assays for this kind of resistance.

A resistance with no direct association with the target (non-target site resistance) can be attributed to all other mechanisms, such as, for example, slowed uptake of the control agent by the pathogen.

Early detection of target site resistance in particular is made possible by the present invention.

The pathogens/infectious agents that are the focus of the present invention attack especially plants, preferably cultivated plants. The term “cultivated plant” is understood to mean a plant which is specifically grown as a useful plant by human intervention. Parts of the cultivated plant which has been grown may be suitable for human and/or animal consumption.

It is in principle conceivable to apply the teaching of the present invention to animals, especially useful animals, as well. The present invention therefore further provides for the application of the teachings discussed in this description to animals, especially useful animals.

In a first step, at least one sample is collected. The term “collecting” is not to be understood as limiting in any way. An example of a synonymous term is the term “sampling”.

The sample is taken from the plant or from the medium in which the plant is growing. The term “medium” shall encompass not only earth, but also any other substrate which is in contact with the plant and which stabilizes the plant and/or supplies it with nutrients. In the case of aquatic plants, a sample can also be taken from the water surrounding the aquatic plant.

The sample collected is preferably a part of the plant, for example a leaf, a flower, a stem, a root, pollen, bark and/or a part of the stated constituents and/or a plurality of the stated constituents and/or other constituents. A sample can also be taken from “secretions” such as nectar or resin. Preferably, various tissue types of the plant are removed and combined in order to cover many different pathogens. Preferably, the plant is in an early stage of development at the time of sampling.

The stage of development of a plant may be stated, for example, in the form of what is called the BBCH code. The abbreviation BBCH officially stands for “Biologische Bundesanstalt, Bundessortenamt und CHemische Industrie” [Federal Biological Research Centre for Agriculture and Forestry, Federal Plant Variety Office, and Chemical Industry]. The extended BBCH scale for uniform codification of the phenological growth stages of mono- and dicotyledonous plants is a collaboration between the Biologische Bundesanstalt für Land- und Forstwirtschaft (BBA) [Federal Biological Research Centre for Agriculture and Forestry], the Bundessortenamt (BSA) [Federal Plant Variety Office], the Industrieverband Agrar (IVA) [Agrochemical Industry Association], and the Institut fur Gemüse u. Zierpflanzenbau Großbeeren/Erfurt [Institute of Vegetable and Ornamental Crops in Grossbeeren/Erfurt] (see, for example, https://www.julius-kuehn.de/publikationsreihen-des-jki/bbch-skala/).

Preferably, the plant is in principal growth stage 1 (leaf development (main shoot)) or principal growth stage 2 (formation of side shoots) or principal growth stage 3 (stem elongation or rosette growth/shoot development (main shoot)) or principal growth stage 4 (development of vegetative plant parts or vegetatively propagated organs/booting) or principal growth stage 5 (inflorescence emergence) or principal growth stage 6 (flowering) of the BBCH scale at the time of sampling. It was found that fungi and/or fungal spores are already detectable in, near or on a plant in these early stages without the plant, however, having to already exhibit visible disease symptoms. Detection at such an early time allows early treatment of the plant in order to prevent an outbreak of a disease and/or the reproduction and/or spread of the pathogens and/or transmission of pathogens to neighbouring plants or to reduce the risk thereof.

Sampling can be done automatically. “Automatically” means that sampling is done by a machine or by multiple machines without any help by a person. Such a machine or device for automatic sampling is also referred to as “sampling unit” in this description.

Sampling can be done with the aid of a mobile device or by a mobile device which, for example, moves or is moved in a field for cultivated plants and/or over the field. For example, it is conceivable to use a (preferably unmanned) land machine and/or a (preferably unmanned) aircraft (e.g. a drone) and/or a robot. In a greenhouse, the use of a robot for automated sampling is ideal.

Sampling can, however, also be done by one or more devices which are fixed at one location.

Furthermore, it is conceivable that a user (person) himself carries out sampling.

The sample is supplied to a sequencing unit. The sequencing unit is configured to determine the sequences of nucleic acids or peptides present in the sample.

“Nucleic acids” are macromolecules which are synthesized from individual building blocks, the nucleotides, and which contain genetic information in the case of organisms and subcellular agents (such as, for example, viruses). In nature, nucleic acids occur in two forms: deoxyribonucleic acid (DNA) and ribonucleic acid (RNA). DNA is the genetic material of all living creatures, from unicellular bacteria to multicellular mammals. Some viruses use RNA instead of DNA as their genetic material. Preferably, DNA strands present in a sample are sequenced.

A “peptide” is an organic compound which contains peptide bonds between amino acids. Long peptide chains are also referred to as proteins. Proteins are found in every cell and are used thereby as molecular “tools” and perform different tasks depending on the particular structure by being able, for example, to allow cell movement, transport metabolites, pump ions, catalyse chemical reactions or recognize signalling substances. The terms “peptide” and “protein” are used synonymously in this description.

The term “sequencing” is understood to mean determining the succession of nucleotides in a nucleic acid or determining the succession of amino acids in a peptide (protein). The sequence is accordingly the succession of nucleotides or the succession of amino acids. The terms “sequence” and “succession” are used synonymously in this description.

It is conceivable that the sample must be prepared before it can be supplied to sequencing. In the case of such preparation, cells in the sample can be disrupted, in order, for example, to release the DNA molecules and then to extract and possibly fragment them. Relevant preparation measures are described in the prior art (see, for example, R. P. Schaudies (Ed.): Biological Identification, Woodhead Publishing Series in Electronic and Optical Materials: Number 59, Elsevier 2014, ISBN 978-0-85709-501-5; Jianping Xu: Next-generation Sequencing, Caister Academic Press 2014, ISBN 978-1-908230-33-1; Vijai Bhadauria: Next-generation Sequencing and Bioinformatics for Plant Science, Caister Academic Press 2017, ISBN 978-1-910190-65-4). For sample preparation, use can be made of a sample preparation unit, which can be a component of the sampling unit and/or the sequencing unit or a separate unit.

Sample preparation can involve amplification of specific regions of the nucleic acids present; however, amplification is not absolutely necessary. Amplification may be appropriate in the case of a region which is characteristic of a species or which can be used to unambiguously and/or reliably identify a species. An example of such a region is the ITS region.

The ITS region (ITS: Internal Transcribed Spacer) is a nucleotide sequence commonly used in phylogenetic analyses. The ITS region is an intergenic, non-coding region of highly variable sequence; it is therefore particularly suitable for determining relationships at the lower taxonomic level of species, genera and families. A sequence analysis of this region can be especially used for identifying the genus and species of fungi (see, for example, T. M. Pryce et al.: Rapid identification of fungi by sequencing the ITS 1 and ITS2 regions using an automated capillary electrophoresis system; Medical Mycology October 2003, 4 1, 369-381; R. H. Nilsson et al.: A Comprehensive, Automatically Updated Fungal ITS Sequence Dataset for Reference-Based Chimera Control in Environmental Sequencing Efforts, Microbes Environ. 2015 June; 30(2): 145-150; A. J. Buehler et al.: Internal transcribed spacer (ITS) sequencing reveals considerable fungal diversity in dairy products, Dairy Sci. 2017; 100(11): 8814-8825; C. L. Schoch et al.: Nuclear ribosomal internal transcribed spacer (ITS) region as a universal DNA barcode marker for Fungi, PNAS Apr. 17, 2012 109 (16) 6241-6246). Comparison of the ITS sequence ascertained and the 5.8S rRNA gene sequence of a fungal strain to be identified makes it possible to determine sequence similarities which serve as a basis for assignment to a taxonomic group. The nucleotide sequence of the ITS region is about 500-600 base pairs. Besides highly conserved regions in which the sequence is identical for all fungi, there are also variable regions. Said variable regions can, as signatures, be characteristic of a species, genus or group of fungi. The analysis of these partial sequences therefore allows taxonomic assignment of fungal strains.

Since the ITS region occurs repeatedly as a gene family with, in each case, multiple copies tandem-repeated hundreds of times to thousands of times, it is stoichiometrically well over-represented compared to an individual nuclear gene and thus easily accessible for molecular phylogenetic analyses. Another advantage of using the ITS region for molecular phylogenetic analyses arises from its position. The ITS region lies in the rRNA gene cluster. It consists of two sequence segments, the Internal Transcribed Spacer 1 (ITS-1) and the Internal Transcribed Spacer 2 (ITS-2), which lie between the highly conserved DNA genes 26S, 5.8S and 18S. This position means that rDNA sequence segments of the highly conserved rDNA genes can be used as primer sequences. Amplification of the ITS-1 and ITS-2 sequences by means of PCR is therefore easily possible.

The sequencing of nucleic acids or peptides present in the sample is performed by the sequencing unit. Preferably, the sequencing is done by means of nanotechnology-based methods (see, for example: M. Loose et al: Real-time selective sequencing using nanopore technology, Nature Methods 2016, Vol. 13 No. 9, 751-758). In the case of nanopore sequencing, nucleic acids for example are individually transported by an enzyme (helicase) through a nanometre-sized pore present in a non-conductive polymer membrane. If a voltage is applied across this membrane, the result is an ion flow through the pore, the size of which ion flow is dependent on the bases in the pore. Therefore, by measuring the ion current during the translocation of the DNA/RNA through the pore, it is possible to measure both the succession of bases and any modifications to the bases.

The sequencing according to the invention can be done in at least two phases, a first phase and a second phase. The first phase serves to clarify whether a pathogen is present at all in the sample. Only then, when a pathogen is identified in the first phase, does the second phase take place. The second phase serves to identify signs of a resistance of the identified pathogen to one or more control agents. In both phases, nucleic acids or peptides are sequenced and successions of nucleotides and succession of amino acids are thereby ascertained. In both phases, the ascertained sequences are compared with reference sequences while being ascertained. In the first phase, the ascertained successions are compared with at least one pathogen sequence. Preferably, a comparison is made with a plurality of pathogen sequences. If there is a match between an ascertained succession of nucleotides or a succession of amino acids and a pathogen sequence, it can be assumed that the pathogen is present in the sample. In this way, a pathogen which is present in the sample can be ascertained. In the second phase, the ascertained successions are compared with at least one resistance marker, which has been observed in the past in the ascertained pathogen, and/or with a resistance-free sequence. This comparison serves to identify signs of the presence of a resistance of the ascertained pathogen to one or more control agents. A resistance-free sequence is a succession of nucleotides or a succession of amino acids of a pathogen that does not exhibit signs of a resistance of the pathogen to a control agent. The pathogen sequence, the resistance marker and the resistance-free sequence are reference sequences which, for example, can be found in one or more databases.

A closer look shall be taken at the first phase first of all.

Most of the nucleic acids or peptides present in a sample can usually be attributed to the plant, especially if the sample is a constituent of a plant. However, determining the sequence of a plant nucleic acid or a plant protein is of secondary interest. Therefore, according to the invention, the sequencing of a nucleic acid or a peptide in the first phase of sequencing is aborted once it is established that the nucleic acid or the peptide does not originate from a pathogen (but from the plant for example).

To this end, the sequencing results are compared with one or more pathogen sequences during sequencing. Thus, while the succession of nucleotides in a nucleic acid or the succession of amino acids in a peptide is being established, a check is made in each case as to whether the succession of nucleotides or the succession of amino acids is present in one or more pathogen sequences. This shall be explained using an example. A first nucleotide in a nucleic acid is first determined, then the second, and so forth. The succession of nucleotides grows with each further nucleotide ascertained.

When a further nucleotide in the succession has been determined, a check is made as to whether the (growing) succession is present in one or more pathogen sequences. It is highly likely that a succession of, for example, four nucleotides will be present at one or more sites in a pathogen sequence. However, in the case of a succession of twenty nucleotides, it may already be the case that said succession no longer occurs at any site in the pathogen sequence. Once it is established that a succession of nucleotides that is ascertained in the sequencing is not present at any site in one or more pathogen sequences, the sequencing can be ended; any determination of a further nucleotide in the succession of nucleotides is then meaningless; it is already clear that the nucleic acid currently being sequenced cannot be a nucleic acid of a pathogen. This also applies analogously to the determination of the succession of amino acids in a peptide in the sample.

The at least one pathogen sequence used for comparison in the first phase of sequencing is a nucleotide sequence or amino acid sequence of a defined pathogen that is specific for the pathogen (or a defined group of pathogens, for example pathogens of a species or genus or sub-family or family or sub-order or order or class). From such a specific pathogen sequence, it is thus possible to infer a specific pathogen (or a group of pathogens). Such a pathogen sequence can, for example, be a nucleic acid sequence which comprises the ITS region in full or in part. In any case, the pathogen sequence must be a sequence which differs from sequences of the plant that is the object under examination.

In a preferred embodiment, one or more pathogen sequences are (automatically) selected on the basis of information relating to the plant, relating to the time of sampling, relating to the site of sampling, relating to the environmental conditions (temperature, air pressure, air humidity, rainfall, etc.) at the time of sampling and/or during a defined time span before the time of sampling (e.g. weather data and/or climate data) and/or relating to history (e.g. pathogens and/or diseases observed in the past). For example, some diseases only occur with certain plants. If a sample is taken from a certain plant, pathogen sequences can be selected from those pathogens which attack said plant and can cause the diseases. Furthermore, pathogens usually occur only in certain regions and only under certain environmental conditions. The site of sampling provides information about the region and about the pathogens that usually occur in the region. The environmental conditions at the time of sampling and/or within a defined time span (e.g. four weeks or three weeks or two weeks or one week or a number of days) before the time of sampling provide information as to the pathogens that should even be taken into account and the pathogens that can be excluded. Those pathogens which have already been observed (either directly or indirectly on the basis of, for example, disease symptoms) in the past at the site of sampling or in the vicinity can also currently occur (if allowed by the environmental conditions and by the plant currently looked at), and this is why a specific pathogen sequence of such a pathogen can (and should) be selected, since it (the pathogen) already occurred in the past.

A preferred embodiment of the present invention therefore comprises the following steps:
(1) ascertaining a plant
(2) ascertaining at least one pathogen which can attack the plant (which has attacked such a plant in the past)
(3) ascertaining at least one pathogen sequence for the at least one ascertained pathogen
(4) collecting a sample of the plant or of a medium in which the plant is growing
(5) identifying a pathogen in the sample
(6) ascertaining at least one resistance marker for the identified pathogen
(7) identifying signs of a resistance of the identified pathogen to a control agent.

A further preferred embodiment of the present invention comprises the following steps:
(1) ascertaining a plant
(2) ascertaining environmental conditions to which the plant is exposed and/or has been exposed
(3) ascertaining at least one pathogen which can attack the plant (which has attacked such a plant in the past)
(4) ascertaining at least one pathogen sequence for the at least one ascertained pathogen
(5) collecting a sample of the plant or of a medium in which the plant is growing
(6) identifying a pathogen in the sample
(7) ascertaining at least one resistance marker for the identified pathogen
(8) identifying signs of a resistance of the identified pathogen to a control agent.

The ascertainment of the plant and/or the ascertainment of the environmental conditions can, for example, be done by a user inputting the relevant information into the system according to the invention. The plant can, for example, be specified by a user inputting the name of the plant or the name of the variety of the plant or a code for the plant/plant variety (e.g. according to the International Code of Nomenclature for Cultivated Plants, or ICNCP for short) into the system according to the invention or selecting the relevant information relating to the plant from a list or a menu or selecting a plant from a pictorial representation of the plant (e.g.: a photograph or a graphic), for example on a monitor of the system according to the invention.

It is also conceivable that a user specifies a plant which is being cultivated on a field and optionally additionally specifies the geo-coordinates of the field. The system according to the invention can be configured to use the specified plant and optionally the geo-coordinates to ascertain in a database pathogens which can occur for the plant species. The geo-coordinates can, for example, be used to ascertain current weather data and/or past weather data. It is conceivable that, for pathogens which can occur for the plant, models are selected that use the weather data to calculate a probability that said pathogens currently occur for the specified plant. It is conceivable that a closer look is taken at only those pathogens for which the probability that they currently occur for the specified plant is above a threshold value (minimum probability). The threshold value can be empirically determined or be arbitrarily defined by a user.

A preferred embodiment of the present invention therefore comprises the following steps:
(1) ascertaining a plant
(2) ascertaining at least one pathogen which can attack the plant (which has attacked such a plant in the past)
(3) ascertaining environmental conditions to which the plant is exposed or has been exposed,
(4) calculating a probability of the at least one pathogen occurring currently for the plant
(5) comparing the probability with a predefined threshold value
(6) ascertaining pathogen sequences for those ascertained pathogens for which the probability is above the threshold value
(7) collecting a sample of the plant or of a medium in which the plant is growing
(8) identifying a pathogen in the sample
(9) ascertaining at least one resistance marker for the identified pathogen,
(10) identifying signs of a resistance of the identified pathogen to a control agent.

Preferably, the one or more pathogen sequences are present as an FM-index. An FM-index is a compressed full-text substring index which is based on the Burrows-Wheeler transform and was developed by P. Ferragina and G. Manzini (see, for example: P. Ferragina and G. Manzini: Opportunistic Data Structures with Applications, Proceedings of the 41st Annual Symposium on Foundations of Computer Science 2000; 390-398). The name FM-index stands for Full-text index in Minute space. The FM-index is an opportunistic data structure which allows both compression of the input text and fast substring query. An FM-index can be used to efficiently ascertain the number of occurrences of a pattern within the compressed input text and to locate the position of each occurrence. The query time and the required storage space has a sublinear complexity with respect to the size of the input data.

The comparison of the succession of nucleotides or succession of amino acids just ascertained with one or more pathogen sequences can be performed by a control and calculation unit. Such a control and calculation unit can be one or more computers. The control and calculation unit can be a component of the sequencing unit or a separate unit. The control and calculation unit has access to one or more data stores. Stored in the at least one data store is at least one pathogen sequence. The control and calculation unit loads the at least one pathogen sequence into a memory, receives the succession of nucleotides during ascertainment thereof (nucleotide by nucleotide or in blocks of multiple nucleotides) and checks whether the (growing) succession of nucleotides is singly or multiply present in the pathogen sequence.

The technology to use a (growing) succession of nucleotides in a sequence during determination thereof for a comparison is described in the prior art (see, for example: S. Kovaka et al.: Targeted nanopore sequencing by real-time mapping of raw electrical signal with UNCALLED, https://doi.org/10.1101/2020.02.03.931923).

Once it is established that the ascertained succession of nucleotides or amino acids does not appear at any site in the at least one pathogen sequence, the sequencing of the respective molecule is aborted and further sequencing of the molecule is thus prevented. The technology to abort determination of a succession of nucleotides is described in the prior art (see, for example: A. Payne et al.: Nanopore adaptive sequencing for mixed samples, whole exome capture and targeted panels, https://doi.org/10.1101/2020.02.03.926956). In the case of nanopore technology, a nucleic acid which is being guided through a pore can be conveyed out of the pore with the aid of a voltage pulse, for example, thereby aborting further sequencing.

If, within a predefined time span, a nucleic acid or peptide in the sample that has a pathogen sequence is not sequenced, it can be assumed that there is no pathogen in the sample. The predefined time span can be empirically determined. A notification indicating that no pathogen was found in the sample can be output.

Once it is established that the sequence of a nucleic acid or of a peptide in the sample matches a specific pathogen sequence, it can be assumed that the corresponding pathogen is present in the sample. Knowledge of the pathogen sequence thus automatically yields the respective pathogen. For the pathogen thus ascertained, a database is used to check whether there are known resistances to one or more control agents. “Known” means that a relevant resistance has been observed in the past in a population of the pathogen. If at least one resistance has been observed, at least one resistance marker is ascertained.

A “resistance marker” is understood to mean genetic information which provides information as to whether a harmful organism might develop, is developing or has developed a resistance to a control agent.

For a multiplicity of pathogens, it is known what sequences indicate that the pathogens develop or have a resistance to a control agent (see, for example: S. Omrane et al.: Plasticity of the MFS1 Promoter Leads to Multidrug Resistance in the Wheat Pathogen Zymoseptoria tritici, https://doi.org/10.1128/mSphere.00393-17; Y. Choi et al.: Identification of Genes Related to Fungicide Resistance in Fusarium fujikuroi, Mycobiology. 2017, 45(2): 101-104; K. J. Brent: Fungicie Resistance in crop pathogens: How can it be managed? ISBN 90-72398-07-6; K. G. Zulak et al.: Improved Detection and Monitoring of Fungicide Resistance in Blumeria graminis f sp. hordei With High-Throughput Genotype Quantification by Digital PCR, Front Microbiol. 2018, 9: 706; M. de miccolis et al: Genetics of Fungicide Resistance, DOI: 10.1007/978-4-431-55642-8_2; H. B. Deising et al.: Mechanisms and significance of fungicide resistance, Braz. J. Microbiol. 2008, Vol. 39 No. 2; L. Kanetis at al.: Fungicide resistance profile and genetic structure of Botrytis cinerea from greenhouse crops in Cyprus, Eur. J. Plant. Pathol. 2017, 147, 527-540; J. L. Beckerman: Detection of Fungicide Resistance, DOI: 10.5772/55981; L. Yang et al.: Cross-resistance of the pathogenic fungus Alternaria alternata to fungicides with different modes of action, BMC Microbiol 2019, 19, 205).

A “resistance marker” is thus a sequence of nucleotides or a sequence of amino acids which was found in nucleic acids or peptides of resistant populations, and which is responsible for the resistance or promotes the resistance. It can, for example, be the target sequence in a resistant pathogen, which target sequence has a mutation compared to non-resistant species of the same species. What is meant by the “target” is the target of the control agent in the pathogen. A genetic modification of the target sequence can cause the control agent to no longer have an effect or to have only a reduced effect.

If there is no known resistance for the identified pathogen, a notification indicating that a pathogen has been identified in the sample, what pathogen it is and that there is no known resistance for the pathogen can be output.

Once a pathogen has been identified in the sample, and at least one resistance marker for the pathogen has been ascertained, the second phase of sequencing is initiated.

In the second phase, the sequencing of the nucleic acids or peptides present in the sample is continued; however, in the second phase, the focus is on identifying signs of a resistance of the identified pathogen to one or more control agents. To this end, the successions of nucleotides or amino acids ascertained in the sequencing are compared with the at least one resistance marker and/or with a resistance-free sequence.

If successions of nucleotides or amino acids that match the sequence of the at least one resistance marker do not occur in the sequencing, it can be assumed that the pathogen identified in the sample does not have a known resistance. A notification indicating that a pathogen was found, what pathogen it is and that no signs of the presence of a known resistance to a control agent were found can be output. However, if deviations occur compared to the resistance-free sequence, it may be that a (yet) unknown resistance is present and/or that the pathogen is developing a (yet unknown) resistance. In that case, a notification can be output that indicates that a pathogen was found, what pathogen it is and that no signs of the presence of a known resistance to a control agent were found, but that deviations are present in the succession of nucleotides or amino acids compared to the resistance-free sequence that can/should be looked at more closely.

If successions of nucleotides or amino acids that match the sequence of a resistance marker do occur in the sequencing, this is a sign of the presence of a (known) resistance. A notification can be output that indicates that a pathogen was found in the sample, that the pathogen has signs of a resistance and that the resistance can be named, for example on the basis of the matching resistance marker.

In a preferred embodiment, the step of “identifying signs of a resistance of the identified pathogen to a control agent” thus comprises the following partial steps:
(1) comparing the sequences of the nucleic acids or peptides in the sample with the at least one ascertained resistance marker and/or a resistance-free sequence,
(2) if the resistance marker does not occur in any of the sequences of the nucleic acids or peptides in the sample and sequences of the sample correspond to the resistance-free sequence: outputting a notification indicating that no sign of a resistance was found for the identified pathogen,
(3) if the resistance marker does not occur in any of the sequences of the nucleic acids or peptides in the sample and the sequences of the sample do not correspond to the resistance-free sequence: outputting a notification indicating that no sign of a known resistance was found for the identified pathogen, but that deviations compared to the resistance-free sequence were found,
(4) if the resistance marker occurs in one or more of the sequences: outputting a notification indicating that a sign of a resistance was found for the identified pathogen, and outputting a notification about the resistance.

It is conceivable that the sequence of a nucleic acid or a peptide of a resistant pathogen differs only by one nucleotide or a few nucleotides or only by one amino acid or a few amino acids from the corresponding sequence of a non-resistant pathogen. On the other hand, errors can always occur when sequencing individual nucleic acids or peptides. Therefore, if a nucleic acid or peptide having a sequence according to a resistance marker is identified, it is in principle possible that this is an error. Therefore, it is conceivable that the presence of a sign of a resistance is only assumed if a minimum number of nucleic acids or amino acids that has a sequence according to a resistance marker was found.

In a preferred embodiment, the step of “identifying signs of a resistance of the identified pathogen to a control agent” thus comprises the following partial steps:
(1) comparing the sequences of the nucleic acids or peptides in the sample with the at least one ascertained resistance marker,
(2) if the resistance marker does not occur in any of the sequences of the nucleic acids or peptides in the sample or occurs in a number of nucleic acids that is smaller than a predefined minimum number: outputting a notification indicating that no sign of a resistance was found for the identified pathogen,
(3) if the resistance marker occurs in a minimum number of sequences or in a number greater than the minimum number: outputting a notification indicating that a sign of a resistance was found for the identified pathogen, and outputting a notification about the resistance.

In a preferred embodiment of the present invention, the sequences of nucleic acids or peptides in a sample in the second phase of sequencing are compared not (only) with resistance markers, but also with the corresponding sequence of a non-resistant pathogen of the same species. Said sequence is also referred to as resistance-free sequence in this description. The resistance-free sequence is a reference sequence, too.

It is conceivable for nucleic acids or peptides in the sample to be found that originate from a pathogen where their sequence does not, however, correspond either to the resistance marker or to the resistance-free sequence. It may be that a new mutation is concerned that might possibly lead to a yet unknown resistance. A comparison of the sequences of the nucleic acids or peptides in the sample with the resistance-free sequence thus makes early identification of new, possibly developing resistances possible.

A preferred embodiment of the present invention therefore comprises the following steps:
collecting a sample of a plant or of a medium in which the plant is growing
identifying a pathogen in the sample
ascertaining at least one resistance marker for the identified pathogen and ascertaining a resistance-free sequence for the identified pathogen
identifying signs of a resistance of the identified pathogen to a control agent by comparison of the sequences of nucleic acids or peptides in the sample with the at least one ascertained resistance marker and the resistance-free sequence
if a sequence or a number of sequences that is greater than or equal to a minimum number does not have a match with either the at least one resistance marker or the resistance-free sequence: outputting a notification indicating that a pathogen having a mutation pointing to a new and/or developing resistance was identified in the sample.

If a pathogen was detected in a sample, but there is no known resistance for the pathogen, the second phase of sequencing can also be initiated, for example in order to analyse the target sequence in the pathogen and to possibly identify mutations pointing to a developing resistance. In such a case, the ascertained successions of nucleotides or amino acids are not compared with the resistance marker, but with the target sequence. The target sequence is a reference sequence, too. The target sequence, the resistance-free sequence and the reference marker are also referred to as resistance-indicating sequence in this description, since a resistance or the absence of a resistance can be established on the basis of this sequence segment.

A preferred embodiment of the present invention therefore comprises the following steps:
collecting a sample of a plant or of a medium in which the plant is growing
identifying a pathogen in the sample
ascertaining a target sequence for the identified pathogen
identifying signs of a new resistance of the identified pathogen to a control agent by comparison of the sequences of nucleic acids or peptides in the sample with the target sequence
if a sequence or a number of sequences that is greater than or equal to a minimum number represents a mutation of the target sequence: outputting a notification indicating that a pathogen having a mutation pointing to a new and/or developing resistance was identified in the sample.

In a preferred embodiment, the frequency Q_{R} of nucleic acids or peptides found in a sample that have a sequence according to the resistance marker is ascertained, and the frequency Q_{NR} of nucleic acids or peptides found in the sample that have a sequence according to the resistance-free sequence is ascertained. The ascertained frequencies Q_{R} and Q_{NR} can be used to form a ratio (e.g. Q_{R}/Q_{NR}). It is conceivable that the presence of a sign of a resistance is assumed only if the ratio has exceeded a predefined minimum value. The minimum value can be empirically determined.

A preferred embodiment of the present invention therefore comprises the following steps:
collecting a sample of a plant or of a medium in which the plant is growing
identifying a pathogen in the sample
ascertaining at least one resistance marker for the identified pathogen and ascertaining a resistance-free sequence for the identified pathogen
comparing the sequences of nucleic acids or peptides in the sample with the at least one ascertained resistance marker and the resistance-free sequence
ascertaining a frequency Q_{R} of nucleic acids or peptides found in the sample that have a sequence according to the resistance marker, ascertaining a frequency Q_{NR} of nucleic acids or peptides found in the sample that have a sequence according to the resistance-free sequence, calculating a ratio of the ascertained frequencies Q_{R} and Q_{NR}, and comparing the ratio with a predefined minimum value
if the ratio is greater than or equal to the minimum value: outputting a notification indicating that a sign of a resistance was found for the identified pathogen, and outputting a notification about the resistance.

What applies in this description to the comparison of the nucleic acid sequences or peptide sequences with the at least one pathogen sequence can otherwise apply analogously to the comparison of the nucleic acid sequences or peptide sequences with the at least one resistance marker and/or the resistance-free sequence and/or the target sequence:
In order also to carry out the second phase of sequencing as efficiently as possible and in order to prevent most of the sequencing from falling onto nucleic acids or peptides of the plant or non-target sequences of the pathogen, the sequencing of individual nucleic acids or peptides can also be aborted in the second phase of sequencing once it is established that a nucleic acid just sequenced or a peptide just sequenced cannot originate from a target sequence of the pathogen (because the ascertained succession does not occur in any pathogen target sequence).
The at least one resistance marker and/or the resistance-free sequence are preferably present as an FM-index.
The control and calculation unit can load the at least one resistance marker and/or the resistance-free sequence into a memory, and receive the (growing) succession of nucleotides or succession of amino acids during ascertainment thereof and compare said succession with the at least one resistance marker and/or with the resistance-free sequence.

Output of information and/or notifications when carrying out the present invention can, for example, be achieved in the form of images and/or text and/or language. Information and/or notifications can be displayed on a screen (monitor), printed out via a printer, stored in a data store and/or transmitted to another computer via a network.

An item of information about a pathogen which has been identified can, for example, be the name of the pathogen. Information indicating the sample in which the pathogen was found and/or the site at which the sample was collected and/or the time at which sampling was carried out and/or the plant from which the sample was taken can also be output.

An item of information about a resistance which has been identified can include the name of the resistance, the target affected by the resistance and/or the name of one or more control agents or one or more active ingredients to which the identified pathogen is resistant.

The result of the analysis of the sample of a plant, which analysis is according to the invention, can be used in various ways. If a pathogen has been identified for which no signs of the presence of a resistance were found or for which the appearance of a new resistance was found, one or more control agents which can be used to control the pathogen can be named. If a pathogen has been identified for which signs of the presence of a known resistance were found, control agents for which the resistance has no effect can be named. Such a control agent can then also be the use of electrical energy, thermal energy (e.g. in the form of steam or flame treatment), electromagnetic radiation (UV radiation, laser irradiation) and/or mechanical means for removing the attacked plant or plant parts.

Since the invention can also be carried out on-site in a field by an autonomous system, the system according to the invention can also comprise a control device for controlling pathogens that, after (automatic) selection of the control agent for controlling the identified pathogen, can automatically apply the selected control agent.

The result of the analysis for pathogens and resistances, which analysis is according to the invention, can furthermore be recorded in a pathogen map. Such a “pathogen map” is a representation of part of the Earth's surface, in which information is recorded for a plurality of locations on the Earth's surface as to whether a pathogen has been observed at the relevant location and whether a sign of an (existing or developing or possible) resistance to a control agent was found for the pathogen. Preferably, the pathogen map is a digital pathogen map.

The term “digital” means that the map can be processed by a machine, generally a computer system.

“Processing” is understood to mean the known methods for electronic data processing (EDP).

Preferably, the digital pathogen map is a digital representation of a field or of a field including adjacent fields or of a region. Preferably, separate pathogen maps are produced for individual pathogens and/or for individual control agents or groups of control agents which comprise the same active ingredient or are of the same chemical/biological class (e.g. a chemical structural class) of active ingredients or have the same mechanism of action or have the same target, and/or for specific plants for which the pathogen can occur. Preferably, separate digital pathogen maps can be combined with one another, i.e. virtually superimposed.

In a preferred embodiment, there is/are stored for each location on the digital pathogen map for which one or more analysis results are available the time or times at which the respective analysis was carried out.

In a preferred embodiment, multiple digital pathogen maps are combined with one another such that they show a chronological development of the spread of one or more resistances.

Preferably, digital pathogen maps can be combined with other digital maps; for example with digital maps relating to soil condition, relating to water balance, relating to cultivated plants that have been grown, relating to temperatures (at defined times and/or for defined time spans, for example in the form of average and/or minimum and/or maximum temperatures), relating to rainfall (at defined times and/or for defined time spans, for example in the form of average and/or minimum and/or maximum rainfall), relating to solar radiation, relating to movements of air mass (wind directions and wind speeds), relating to attacks that occurred in the past by one or more pathogens, relating to agricultural measures which have been taken (e.g. sowing, irrigation, ploughing, application of plant protection products, administration of nutrients, and the like), etc. The values of the parameters recorded in a digital map can be measured and/or predicted values.

A pathogen map can, for example, provide a farmer with valuable information as to the regions of his field (or fields) in which pathogens are present and the resistances which are present or spreading. Spread patterns can be analysed in order to find out the causes of the emergence and/or spread of pathogens and/or resistances. With multiple analyses following one another over time, the farmer gains an insight into the spread of pathogens and/or resistances. Said farmer can then take measures to control the pathogens and/or resistances. In a pathogen map, a farmer can also identify whether an attack by (resistant) pathogens has been observed in adjacent fields. The information can help said farmer to take preventive measures for his fields.

Furthermore, detailed digital pathogen maps generated almost in real-time, in combination with further data, such as, for example, solar radiation, temperature, air humidity and wind direction, allow a more accurate prediction concerning the spread of resistant and non-resistant pathogens. This allows specific control, or prophylactic treatment, of adjoining areas, and more specific use of control agents.

Furthermore, the specific recording of genetic information of pathogen populations in the environment makes it possible to assess an imminent development of resistance to a particular control agent.

In a further step of the method according to the invention, a measure to control a pathogen and/or a (developing) resistance can be recommended and/or carried out (on the basis of the pathogen map). First of all, one or more control agents for controlling the pathogen can be identified on the basis of the pathogen which has been identified in the sample. This can, for example, be done by a query in one or more databases. Stored in the one or more databases can be information about control agents and about the pathogens against which they are used. If the identified pathogen has a resistance, a check can be made in a second step as to whether the resistance is to one or more of the control agents ascertained from the at least one database. What can be identified is that control agent or those control agents to which the identified pathogen is not resistant. The plants can then be treated with the (one of the) identified control agent(s). If the pathogen should be resistant to all the control agents ascertained from the at least one database, alternative measures for controlling the pathogens and/or spread of a disease can then be identified, proposed and/or carried out, such as, for example, complete mechanical removal of the affected plants, crop rotation, flame treatment, cold destruction, destruction by electrical energy, destruction by electromagnetic energy (e.g. by means of UV light, laser light) and/or the like.

The present invention can be performed wholly or partly with a computer system. A “computer system” is an electronic data processing system that processes data by way of programmable computing rules. Such a system usually comprises a control and calculation unit, often also referred to as “computer”, said unit comprising a processor for carrying out logical operations and a memory for loading a computer program, and also peripherals.

In computer technology, “peripherals” refers to all devices that are connected to the computer and are used for control of the computer and/or as input and output devices. Examples thereof are monitor (screen), printer, scanner, mouse, keyboard, joystick, drives, camera, microphone, speakers, etc. Internal ports and expansion cards are also regarded as peripherals in computer technology.

Modern computer systems are frequently divided into desktop PCs, portable PCs, laptops, notebooks, netbooks and tablet PCs, and what are called handhelds (e.g. smartphones); all these systems can be utilized for execution of the invention.

Inputs into the computer system (e.g. for control by a user) are achieved via input means such as, for example, a keyboard, a mouse, a microphone, a touch-sensitive display and/or the like. Outputs are achieved via one or more output units, which may be especially a monitor (screen), a printer and/or a data store.

The invention will be more particularly elucidated below on the basis of specific exemplary embodiments and figures, without wishing to restrict the invention to said examples and to the features and/or combinations of features shown in the examples.

In the figures:

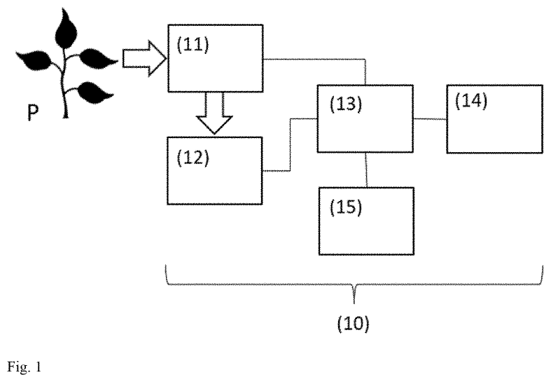

FIG. 1 shows schematically one embodiment of the system according to the invention. The system (10) comprises a sampling unit (11), a sequencing unit (12), a control and calculation unit (13), a data store (14) and an output unit (15).

By means of the sampling unit (11), a sample can be taken from a plant P or from a medium in which the plant P is growing. The sample can be prepared and be supplied to the sequencing unit (12).

By means of the sequencing unit (12), sequences of nucleic acids present in the sample are ascertained.

The control and calculation unit (13) is used to control the individual components of the system (10) according to the invention and to coordinate the data and signal flows. The control and calculation unit can be a general-purpose computer which has been appropriately configured by means of the computer program according to the invention to execute the steps described here.

The control and calculation unit (13) is connected to the data store (14), from which it can retrieve one or more pathogen sequences and one or more resistance-indicating sequences for comparative purposes.

The control and calculation unit (13) receives growing sequences of nucleic acids from the sequencing unit (12).

In a first phase, the control and calculation unit (13) checks whether the growing sequences are present in one or more pathogen sequences. Once it is established that a growing sequence of a nucleic acid is not present in the at least one pathogen sequence, the control and calculation unit (13) sends a signal to the sequencing unit (12). Owing to the signal, the sequencing of the nucleic acid is aborted, and the sequencing of a new nucleic acid is started. Once it is established that the sequence of a nucleic acid matches a pathogen sequence, the control and calculation unit (13) prompts the output unit (15) to output information about the pathogen having the sequence matched by the sequence of the nucleic acid. Furthermore, the control and calculation unit (13) switches to a second phase.

In the second phase, the control and calculation unit (13) checks whether the growing sequences of the nucleic acids in the sample have a match with a resistance marker, a resistance-free sequence and/or a target sequence. Once it is established that the sequence of a nucleic acid matches a resistance-indicating sequence, the control and calculation unit (13) prompts the output unit (15) to output information about the resistance.

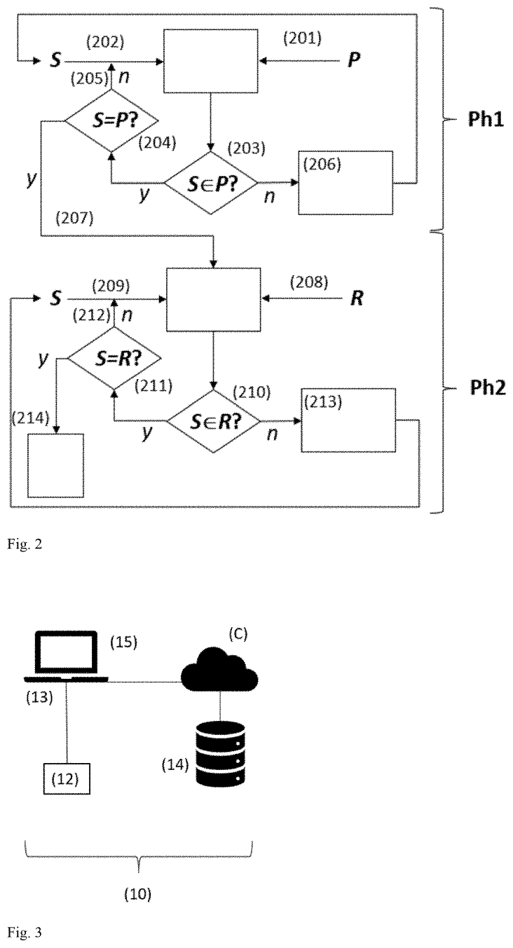

FIG. 2 shows, schematically and by way of example in the form of a flow chart, the steps executed by a computer that has loaded in the memory thereof the computer program according to the invention. The steps are divided into two phases Ph1 and Ph2. Steps (201), (202), (203), (204), (205) and (206) are assigned to the first phase. Steps (208), (209), (210), (211), (212), (213) and (214) are assigned to the second phase. Step (207) is assigned to the first phase and leads to a switch from the first phase Ph1 to the second phase Ph2. The computer starts with the execution of the steps in phase Ph11 and switches to phase Ph2 when defined conditions are present (see below).

(201) The computer loads multiple pathogen sequences P from a data store.

(202) The computer receives a growing sequence S of a nucleic acid.

(203) The computer checks whether the growing sequence S is present in the pathogen sequences P (S∈P?).

(206) If the growing sequence S is not present in any of the pathogen sequences P, the computer sends a signal which leads to the abortion of the sequencing of the currently sequenced nucleic acid. Instead, a new nucleic acid is sequenced and the computer receives a growing sequence S of the new nucleic acid.

(204) If a growing sequence S is present in a pathogen sequence P, a check is made as to whether it can be assumed on the basis of the available information that the sequence S can be assigned to a specific pathogen from P (S=P?).

(205) If the available information is not yet sufficient to state that the sequence originates from a specific pathogen, the sequencing of the nucleic acid is continued.

(207) If the available information is sufficient to state that the sequence does originate from a specific pathogen, the pathogen in the corresponding sample is identified. An item of information about the pathogen can be output, and the computer sends a signal which leads to the abortion of the sequencing of the currently sequenced nucleic acid. Furthermore, a switch is made to the second phase Ph2 of sequencing.

(208) The computer loads at least one resistance-indicating sequence R for the identified pathogen from a data store, wherein the at least one resistance-indicating sequence can include a resistance marker, a resistance-free sequence and/or a target sequence.

(209) The computer receives a new growing sequence S of a nucleic acid.

(210) The computer checks whether the growing sequence S is present in the at least one resistance-indicating sequence R (S∈R?).

(213) If the growing sequence S is not present in any resistance-indicating sequence, the computer sends a signal which leads to the abortion of the sequencing of the currently sequenced nucleic acid. Instead, a new nucleic acid is sequenced and the computer receives a growing sequence S of the new nucleic acid.

(211) If the growing sequence S is present in the resistance-indicating sequence R, a check is made as to whether it can be assumed on the basis of the available information that the pathogen has the corresponding resistance (S=R?).

(212) If the available information is not yet sufficient to state that the pathogen has the resistance, the sequencing of the nucleic acid is continued.

(214) If the available information is sufficient to state that the pathogen has the present resistance, an item of information about the resistance is output.

FIG. 3 shows schematically another embodiment of the system according to the invention. The system (10) comprises a sequencing unit (12), a control and calculation unit (13), a data store (14) and an output unit (15).

The control and calculation unit (13) and the output unit (15) are components of a standard computer system, for example a laptop. The control and calculation unit (13) receives data from the sequencing unit (12), said data representing growing successions of nucleotides or amino acids. Furthermore, the control and calculation unit (13) is connected to the database (14) via a network (e.g. the Internet, represented here by a cloud (C)). It is conceivable that multiple databases are present. It is also conceivable that a database is a component of the computer system. From the at least one database, the control and calculation unit (13) obtains at least one pathogen sequence with or without at least one resistance-indicating sequence.

It is conceivable that the at least one database can also provide data relating to environmental conditions, data relating to control agents, data relating to plants and plant varieties, data relating to pathogen and/or the like. Furthermore, it is conceivable that one or more models for calculating the probability of a pathogen occurring currently for a plant on the basis of information relating to the plant and/or relating to the environmental conditions are stored in the at least one database, which model(s) can be retrieved by the control and calculation unit (13) and used for calculation purposes.

1. A method comprising the steps of:
collecting a sample of a plant or of a medium in which the plant is growing;
identifying a pathogen in the samples;
identifying signs of a resistance of the pathogen to a control agent;
wherein the identification of the pathogen and the identification of the signs of a resistance is done by sequencing individual nucleic acids or individual peptides in the sample, wherein sequences of nucleotides or sequences of amino acids are ascertained in the sequencing of the individual nucleic acids or individual peptides, wherein the sequences of nucleotides or sequences of amino acids are compared with reference sequences, wherein the sequencing is done in two phases, a first phase and a second phase;
wherein, in the first phase, during the sequencing of each nucleic acid or each peptide, the respectively ascertained sequence is compared with at least one pathogen sequence; and
if the ascertained sequence does not match the at least one pathogen sequence, further sequencing of said nucleic acid or said peptide is aborted;
if the ascertained sequence matches a pathogen sequence, a pathogen having the pathogen sequence is ascertained and the ascertained pathogen is used to ascertain at least one resistance marker, and a switch is made to the second phase;
wherein, in the second phase, sequences of the nucleic acids or peptides in the sample are compared with the at least one ascertained resistance marker and/or with a resistance-free sequence.

2. The method according to claim 1, comprising the steps of:
ascertaining the plant;
ascertaining at least one pathogen which can attack the plant;
ascertaining at least one pathogen sequence for the at least one ascertained pathogen;
collecting the sample of the plant or of the medium in which the plant is growing;
identifying the pathogen in the sample;
ascertaining at least one resistance marker for the identified pathogen; and
identifying signs of resistance of the identified pathogen to a control agent.

3. The method according to claim 1, comprising the steps of:
ascertaining the plant;
ascertaining environmental conditions to which the plant is exposed and/or has been exposed;
ascertaining at least one pathogen which can attack the plant;
ascertaining at least one pathogen sequence for the at least one ascertained pathogen;
collecting the sample of the plant or of the medium in which the plant is growing;
identifying the pathogen in the sample;
ascertaining at least one resistance marker for the identified pathogen; and
identifying signs of resistance of the identified pathogen to the control agent.

4. The method according to claim 1, comprising the steps of:
ascertaining the plant;
ascertaining at least one pathogen which can attack the plant;
ascertaining environmental conditions to which the plant is exposed or has been exposed;
calculating a probability of the at least one pathogen occurring currently for the plant;
comparing the probability with a predefined threshold value;
ascertaining pathogen sequences for those ascertained pathogens for which the probability is above the threshold value;
collecting the sample of the plant or of the medium in which the plant is growing;
identifying the pathogen in the sample;
ascertaining at least one resistance marker for the identified pathogen; and
identifying signs of resistance of the identified pathogen to a control agent.

5. The method according to claim 1, wherein the step of identifying signs of a resistance of the identified pathogen to a control agent comprises the following steps:
comparing the sequences of the nucleic acids or peptides in the sample with the at least one ascertained resistance marker;
wherein if the resistance marker does not occur in any of the sequences of the nucleic acids or peptides in the sample: outputting a notification indicating that no sign of a resistance was found for the identified pathogen; and
wherein if the resistance marker occurs in one or more of the sequences: outputting a notification indicating that a sign of a resistance was found for the identified pathogen, and outputting a notification about the resistance.

6. The method according to claim 1, wherein the step of identifying signs of resistance of the identified pathogen to a control agent comprises the following partial steps:
comparing the sequences of the nucleic acids or peptides in the sample with the at least one ascertained resistance marker;
wherein if the resistance marker does not occur in any of the sequences of the nucleic acids or peptides in the sample or occurs in a number of nucleic acids that is smaller than a predefined minimum number then outputting a notification indicating that no sign of a resistance was found for the identified pathogen;
wherein if the resistance marker occurs in a minimum number of sequences or in a number greater than the minimum number then outputting a notification indicating that a sign of a resistance was found for the identified pathogen, and outputting a notification about the resistance.

7. The method according to claim 1, comprising the steps of:
collecting the sample of a plant or of the medium in which the plant is growing;
identifying the pathogen in the sample;
ascertaining at least one resistance marker for the identified pathogen and ascertaining a resistance-free sequence for the identified pathogen; and
identifying signs of resistance of the identified pathogen to a control agent by comparison of the sequences of nucleic acids or peptides in the sample with the at least one ascertained resistance marker and the resistance-free sequence;
wherein if a sequence or a number of sequences that is greater than or equal to a minimum number does not have a match with either the at least one resistance marker or the resistance-free sequence then outputting a notification indicating that a pathogen having a mutation pointing to a new and/or spreading resistance was identified in the sample.

8. The method according to claim 1, comprising the steps of:
collecting the sample of a plant or of the medium in which the plant is growing;
identifying the pathogen in the sample;
ascertaining a target sequence for the identified pathogen; and
identifying signs of a new resistance of the identified pathogen to a control agent by comparison of the sequences of nucleic acids or peptides in the sample with the target sequence;
wherein if a sequence or a number of sequences that is greater than or equal to a minimum number represents a mutation of the target sequence then outputting a notification indicating that a pathogen having a mutation pointing to a new and/or spreading resistance was identified in the sample.

9. The method according to claim 1, comprising the steps of:
collecting the sample of a plant or of the medium in which the plant is growing;
identifying the pathogen in the sample;
ascertaining at least one resistance marker for the identified pathogen and ascertaining a resistance-free sequence for the identified pathogen;
comparing the sequences of nucleic acids or peptides in the sample with the at least one ascertained resistance marker and the resistance-free sequence; and
ascertaining a frequency Q_{R} of nucleic acids or peptides found in the sample that have a sequence according to the resistance marker, ascertaining a frequency Q_{NR} of nucleic acids or peptides found in the sample that have a sequence according to the resistance-free sequence, calculating a ratio of the ascertained frequencies Q_{R} and Q_{NR}, and comparing the ratio with a predefined minimum value;
wherein if the ratio is greater than or equal to the minimum value then outputting a notification indicating that a sign of a resistance was found for the identified pathogen, and outputting a notification about the resistance.

10. The method according to claim 1, wherein the sample of the plant is a leaf or multiple leaves of the plant.

11. The method according to claim 1, wherein the plant is in principal growth stage 1 or 2 or 3 to 6 according to the BBCH scale.

12. The method according to claim 1, wherein the pathogen sequence is a succession of DNA nucleotides from a fungus or bacterium or virus.

13. The method according to claim 1, further comprising the steps of:
creating and/or updating a pathogen map, wherein a location is recorded in the pathogen map, wherein the sample of the plant has been collected at the location, wherein an indication is given for the location as to whether a pathogen has been identified in the plant and, if a pathogen has been identified in the plant, whether the pathogen has a resistance to a control agent; and
outputting the pathogen map, preferably displaying the pathogen map on a screen and/or outputting the pathogen map on a printer and/or transmitting the pathogen map to a device for automatic execution of a measure to control the identified pathogen.

14. The method according to claim 1, further comprising the steps of:
ascertaining a control agent for controlling the identified pathogen, taking into account the resistance that may have been identified; and
carrying out a measure to control the pathogen in the plant using the ascertained control agent.

15. A system comprising:
a sequencing unit;
a control and calculation unit; and
at least one data store in which at least one pathogen sequence and at least one resistance marker are stored for at least one pathogen;
wherein the sequencing unit is configured to sequence nucleic acids or peptides in a sample of a plant or of a medium in which the plant is growing and to thereby ascertain sequences of nucleotides or amino acids in the sample;
wherein the sequencing unit is configured to transmit, during the ascertainment of the sequences, the respectively ascertained sequences to the control and calculation unit;
wherein the control and calculation unit is configured to compare, in a first phase, each transmitted sequence of a nucleic acid or a peptide with at least one pathogen sequence; and
if a transmitted sequence does not match the at least one pathogen sequence, to prompt the sequencing unit to abort further sequencing of the respective nucleic acid or the respective peptide;
if a transmitted sequence matches a pathogen sequence, to ascertain a pathogen having the pathogen sequence and to use the ascertained pathogen to ascertain at least one resistance marker, and to make a switch to a second phase;
wherein the control and calculation unit is configured to compare, in the second phase, sequences of the nucleic acids or peptides in the sample with the at least one ascertained resistance marker and/or a resistance-free sequence.

16. A non-transitory computer-readable storage medium comprising commands which, upon execution by a computer, prompt said computer to execute the following steps:
for a multiplicity of nucleic acids or peptides: receiving a growing succession of nucleotides or amino acids in the respective nucleic acid or the respective peptide from a sequencing unit;
in a first phase while the growing succession is being received: checking whether the succession of nucleotides or amino acids is singly or multiply present in at least one pathogen sequence;
wherein if the succession is not present in any pathogen sequence then transmitting a signal to the sequencing unit to abort further sequencing of the nucleic acid or the peptide;
wherein if the succession matches a pathogen sequence then ascertaining a pathogen having the pathogen sequence, and ascertaining at least one reference marker, and initiating a second phase; and
in the second phase: comparing the successions of nucleotides or amino acids with the at least one reference marker and/or a resistance-free sequence.
